(19) United States
(12) Patent Application Publication
(10) Pub. No.: US 2010/0008980 A1
(43) Pub. Date: Jan. 14, 2010

(54) Use of MAGE A3-Protein D Fusion Antigen in Immunotherapy Combined with Surgery, Chemotherapy or Radiotherapy for the Treatment of Cancer

(76) Inventors: Vincent Brichard, Rixensart (BE); Catherine Marie Ghislaine Gerard, Rixensart (BE); Frederic Francois Eugene Lahmann, Rixensart (BE); Jamila Louahed, Rixensart (BE)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 12/520,882

(22) PCT Filed: Jan. 8, 2008
(86) PCT No.: PCT/EP08/50133

§ 371 Date: Jun. 23, 2009

(30) Foreign Application Priority Data
Jan. 8, 2007 (GB) 0700284.3

(51) Int. Cl. A61K 9/127; A61K 39/00

(52) U.S. Cl. 424/450; 424/185.1

(57) ABSTRACT

A combination therapy comprising an immunotherapy based on a tumour antigen or an immunological derivative thereof, and at least one other treatment for cancer such as chemotherapy, radiotherapy and/or surgery.

The present invention relates to a combination therapy comprising an immunotherapy based on a cancer testis antigen or an immunological derivative thereof, and at least one other treatment for cancer such as chemotherapy, radiotherapy and/or surgery.

Several gene families have been identified, which encode for so-called cancer/testis antigens. These are antigens/proteins that are generally not expressed in normal tissues/cells other than the testes. However, these antigens are thought to be expressed specifically in certain cancers/tumors such as bladder, breast, lung particularly non-small cell lung cancer (NSCLC), liver, seminomas, melanoma, and/or head and neck cancers and advantageously are capable of being recognised by cytotoxic T-cells.

More than 50 cancer/testis antigens have been described so far and, for many of them, specific epitopes recognized by T lymphocytes have been identified.

A well characterised cancer/testis antigen family is the MAGE family, which includes 12 closely related genes, MAGE 1, MAGE 2, MAGE 3, MAGE 4, MAGE 5, MAGE 6, MAGE 7, MAGE 8, MAGE 9, MAGE 10, MAGE 11, MAGE 12, located on chromosome X and sharing with each other 64 to 85% homology in their coding sequence (De Plaen, 1994). These are sometimes known as MAGE A1, MAGE A2, MAGE A3, MAGE A4, MAGE A5, MAGE A6, MAGE A7, MAGE A8, MAGE A9, MAGE A 10, MAGE A11, MAGE A 12 (The MAGE A family).

Two other groups of proteins are also part of the MAGE family although more distantly related. These are the MAGE B and MAGE C group. The MAGE B family includes MAGE B1 (also known as MAGE Xp1, and DAM 10), MAGE B2 (also known as MAGE Xp2 and DAM 6) MAGE B3 and MAGE B4—the Mage C family currently includes MAGE C1 and MAGE C2.

In general terms, a MAGE A protein can be defined as containing a core sequence signature located towards the C-terminal end of the protein (for example with respect to MAGE A1 a 309 amino acid protein, the core signature corresponds to amino acid 195-279).

The consensus pattern of the core signature is thus described as follows wherein x represents any amino acid, lower case residues are conserved (conservative variants allowed) and upper case residues are perfectly conserved.

### Core Sequence Signature:

LixvL(2x)I(3x)g(2x)apEExiWexl(2x)m(3-4x)Gxe(3-
4x)gxp(2x)Ilt(3x)VqexYLxYxqVPxsxP(2x)yeFLWGprA(2x)
Et(3x)kv

Conservative substitutions are well known and are generally set up as the default scoring matrices in sequence alignment computer programs. These programs include PAM250 (Dayhoft M. O. et al., (1978), “A model of evolutionary changes in proteins”, In “Atlas of Protein sequence and structure” 5(3) M. O. Dayhoft (ed.), 345-352), National Biomedical Research Foundation, Washington, and Blosum 62 (Steven Henikoft and Jorja G. Henikoft (1992), “Amino acid substitution matrices from protein blocks”), Proc. Natl. Acad. Sci. USA 89 (Biochemistry): 10915-10919.

Other cancer/testis antigens include LAGE 1 and LAGE 2.

WO 94/23031 describes MAGE 3. WO 95/20974 describes MAGE 1.

MAGE 3 is thought to be expressed in certain populations of patients with: melanoma, NSCLC and in head and neck cancer.

MAGE C1 and C2 are thought to be expressed in populations of patients with bladder cancer and/or breast cancer.

Lung cancer is the leading a cause of death in many parts of the world. Non-small cell lung cancer constitutes 75-80% of lung cancer cases and accounts for approximately 1.2 million new cases worldwide each year [Parkin, 2001; Jemal, 2005]. For NSCLC, surgery remains the only treatment with curative potential but, unfortunately, only less than one third of all NSCLC patients are suitable for radical surgery at the time of diagnosis. In radically resected NSCLC, cancer reoccurs in more than 80% of cases within two years from the time of surgery.

At the present time adenocarcinoma seems to be the predominant histological subtype. It has a higher propensity for spread of the cancer to a location distant from the initial occurrence. The most common site of metastatic relapse is the brain, followed by bone, lung, liver and adrenal glands [Feld, 1984; Pairolero 1984, Thomas 1990, Martini 1980].

Work has been ongoing to develop cancer treatment based on cancer testis antigens but to date no marketed products have been launched.

In the context of cancer treatment adjuvant therapy refers to chemotherapy or radiotherapy following surgery. For a long period of time, post-operative thoracic radiotherapy has been the preferred adjuvant treatment for patients with NSCLC after resection. Results regarding its potential role have been reported from a number of studies and from PORT (Post-Operative RadioTherapy) meta-analysis [PORT group 1998]. This meta-analysis showed that post-operative chest radiotherapy has, overall, a detrimental effect on survival. Subgroup analyses suggest that this adverse effect is greatest in patients with stage I and II disease, whereas, for those with stage III disease, there is no clear evidence of either a positive or a negative effect.

The role of platinum-based chemotherapy has now been established in advanced-stage NSCLC: stage IV, inoperable stage III (combined with thoracic radiotherapy) and stage IIIA disease (when given before radical surgery) [Spira 2004; Pfister 2004]. Recent trials have suggested a possible role of chemotherapy in prolonging survival of NSCLC patients after complete resection, when chemotherapy is used as an adjuvant treatment. According to the meta-analysis published in 1995 [NSCLC group 1995], which showed a statistically non-significant 5% improvement in 5-year survival with second-generation platinum-based adjuvant chemotherapy. Eight prospective trials addressing the role of adjuvant second- and third-generation platinum-based chemotherapy have been completed [Keller 2000, Scagliotti 2003, Arriagada 2004, Waller 2004, Tada 2004, Winton 2005, Strauss 2004, Douillard 2005].

Three of these trials had a positive outcome, showing a statistically significant reduction in mortality with adjuvant chemotherapy, whereas the outcomes of five others were negative, showing no survival benefit for adjuvant chemotherapy.

In summary, most of the data consistently shows a small reduction in the rate of lung cancer relapse and mortality (an improvement in 5-year absolute survival of 4-15%) with modern adjuvant chemotherapy in complete resected stage II and IIIA disease. These data are more debatable for NSCLC stage IB, especially in view of the last up-date of the CALGB9633 study [Strauss et al. ASCO, 2006].

### Cyclophosphamide

Cyclophosphamide (CY) is a chemotherapeutic agent used to treat various types of cancer. High doses of this drug are required for effective chemotherapy. High doses of CY may lead to immunosuppression while low doses of the drug can lead to enhanced immune responses against a variety of antigens.

It has also been suggested that CY decreases the number of T cells with a phenotype of regulatory T cells (T reg constitutively express CD25: CD4+CD25+).

Regulatory T cells (Treg) control key aspects of tolerance and play a role in the lack of natural anti-tumor immune responses. Indeed, because tumor-associated antigens are derived from self antigens, Treg may be partially responsible for the poor effectiveness of vaccine-induced anti-tumor immune responses. In animals it has been shown that the removal CD4+CD25+ Treg enhances anti-tumor immune responses.

### Dexamethasone

Cancer patients who receive various chemotherapeutic regimes are often pre-treated several days before with anti histaminic (5HT3 receptor antagonists) or/and steroid/glucocorticoids such as dexamethasone to decrease the side effects of the chemotherapeutic agents. Dexamethasone is given either as an anti emetic or to decrease potential allergic reactions.

Glucocorticoids such as dexamethasone have a profound suppressive effect on immune responses through an impact on lymphocytes, inducing their apoptosis and on dendritic cells, inhibiting their expression of CCR7 and their capacity to migrate to the lymph nodes. (Vizzardelli et al, Eur. J. Immunol. 2006 June; 36(6):1504-15.)

The treatment of unresectable stage III NSCLC remains challenging. Standard therapies comprise chemotherapy with sequential thoracic radiotherapy or the administration of these two therapies concurrently. Most recently, induction and consolidation regimens have been evaluated which involve adding chemotherapy before concurrent chemo-/radiotherapy (as induction) or after (as consolidation).

However, this does not appear to make a dramatic impact on overall rates of relapse and survival.

Thus, there is still a large population of patients with cancer that, despite undergoing severe treatment regimes, with significant side effects, that do not see a significant increase in life expectancy and prognosis or for whom no effective treatment exits.

Thus there is still a need in this medical field for alternative therapies that are effective.

### BRIEF DESCRIPTION OF THE FIGURES

FIG. 1—CD4 response: impact of anti-CD25 or cyclophosphamide on the TC1Mage3 therapeutic model

FIG. 2—CD8 response: impact of anti-CD25 or cyclophosphamide on the TC1Mage3 therapeutic model

FIG. 3—In vivo tumor growth of TC1Mage3 cells from day 0 to day 28 (10e5 cells injected)

FIG. 4—Schematic showing experimental protocol for dexamethasone experiments

FIG. 5—Exp 20060590: n=3 mice/group individually treated

FIG. 6—Exp 20060803 n=3 mice/group individually treated)

FIG. 7—Exp 20070129 n=3 mice/group individually treated

FIG. 8—Exp 20060590 n=3 mice/group individually treated

FIG. 9—Exp 20060803 n=3 mice/group individually treated

FIG. 10—Exp 20070129 n=3 mice/group individually treated

FIG. 11—Exp 20060590: CD4 response in spleen (n=3 mice/group individually treated)

FIG. 12—Exp 20060590: CD8 response in spleen (n=3 mice/group individually treated)

FIG. 13—Exp 20060590: CD4 response in PBL (1 pool/group of 3 mice)

FIG. 14—Exp 20060590: CD8 response in PBL (1 pool/group of 3 mice)

FIG. 15—Exp 20060803: CD4 response in spleen (n=3 mice/group individually treated)

FIG. 16—Exp 20060803: CD8 response in spleen (n=3 mice/group individually treated)

FIG. 17—Exp 20060803: CD4 response in PBL (1 pool of 3 mice/group)

FIG. 18—Exp 20060803: CD4 response in PBL (1 pool of 3 mice/group)

FIG. 19—Exp 20070129: CD4 response in spleen (n=3 mice/group individually treated)

FIG. 20—Exp 20070129: CD8 response in spleen (n=3 mice/group individually treated)

FIG. 21—Exp 20070129: CD4 response in PBL (1 pool of 3 mice/group)

FIG. 22—Exp 20070129: CD8 response in PBL (1 pool of 3 mice/group)

FIG. 23: serology (exp 20060590) 14 days post 2 ASCI injection and 18 days after the last dexamethasone injection

FIG. 24: Impact of dexamethasone on the TC1 MAGE3 model/20070129

FIG. 25: Trial design summary

### SUMMARY OF THE INVENTION

The present invention provides a combination therapy comprising administering a therapeutically effective amount of an immunotherapy comprising a tumour antigen or an immunogenic fragment thereof or a fusion protein thereof, and an immunostimulant such as an adjuvant for stimulating a humoral and/or cellular response, wherein said immunotherapy is administered prior, concurrently and/or post:
i) surgery to remove some or all of the cancer said surgery is characterised in that it includes lymph node sampling or complete lymph node dissection (lymphadenctomy), and/or
ii) chemotherapy, and/or
iii) radiotherapy.

In one embodiment of the present invention the tumour antigen is a cancer/testis antigen.

In one aspect the invention provides administering said immunotherapy composition after some or all of the relevant cancer has been removed by the surgery. In this aspect the immunotherapy may be administered concurrently with chemotherapy or radiotherapy. Alternatively, the immunotherapy may be administered subsequent to the adjuvant chemo or radiotherapy. Generally any treatment, such as immunotherapy, will commence within 8 weeks of the surgery.

Lymph node sampling in the context of this specification is intended to refer to removal of all lymph nodes that have been or are highly likely to have been infected by the cancer/tumor, for example as a result of their proximity to the cancer.

The inventors believe from empirical observation that, where appropriate, patients who receive complete lymph node dissection overall may have a better prognosis (survival and relapse rate) than corresponding patients who do not receive this procedure.

Complete lymph node dissection in the context of the present application is intended to refer to the removal of all lymph nodes even those remote from the cancer/tumor.

In another aspect the invention provides, for example where the cancer is inoperable, administering said immunotherapeutic composition after chemotherapy or radiotherapy.

Alternatively, the chemo or radiotherapy may be initiated for a period of, for example 1 to 8 weeks, followed by a treatment regime of chemo or radiotherapy and concomitant administration of the immunotherapeutic composition.

In one aspect the invention comprises chemotherapy, followed by a treatment regime of radiotherapy, followed in turn by a regime comprising administering said immunotherapy, for example wherein the immunotherapeutic regime is maintenance therapy.

Alternatively the immunotherapy may be administered prior to initiation of other forms of treatment such a surgery, chemotherapy and/or radiotherapy. This may assist in shrinking tumors or reducing cancers, which may, for example, facilitate removal of the tumor/cancer by surgery.

It is thought that the combination therapies according to the invention will lead to improved treatments for the relevant patient populations. For example, it may be possible to reduce the dose of chemotherapy or radiotherapy given and thereby reduce the side effects suffered by the patient. Furthermore, it is hypothesised that it will improve the long term survival of and/or prognosis of patients treated with the therapy in comparison to patients who have not been treated with the therapy.

Furthermore, it is thought that where the immunotherapy is combined with chemotherapy that the chemotherapy may stimulate the increased productions of the cancer testis antigens in vivo thereby resulting in a combination treatment with improved efficacy.

The present invention is hoped to benefit patients with cancer antigen expressing cancers such as NSCLC, for example in one or more of the stages defined above or one of the other cancers listed above, such as melanoma. Patients with cancers which are particularly susceptible to metastasis may be particularly benefited.

It appears to the inventors that not all patients who have a cancer/tumour expressing a tissue specific or cancer testis antigen have a clinical response to an appropriate monotherapy employing a relevant immunotherapy comprising a tissue specific or cancer testis antigen as appropriate. It is further thought that patients who respond to the relevant monotherapy have specific genetic profiles, which predispose them to this response. Nevertheless it is believed that by administering a chemotherapeutic agent and/or radiotherapy prior to initiation of the immunotherapy treatment regime may, advantageously, induce patients (who would otherwise be non-responders to the immunotherapy) to become a responder thereto.

Responder in this context includes patients where the cancer/tumour(s) is eradicated, reduced or improved (mixed responder or partial responder) or simply stabilised such that the disease is not progressing. In responders where the cancer is stabilised then the period of stabilisation is such that the quality of life and/or patients life expectancy is increased (for example stable disease for more than 6 months) in comparison to a patient that does not receive treatment.

In one embodiment of the present invention there is provided use of a combination therapy as described herein in the preparation of a medicament for altering the genetic profile of a tumour from a non-responder to be a responder to treatment with an immunotherapeutic agent or combination therapy as described herein.

First line treatment in the context of this specification is intended to refer the first treatment that is initiated for the patient's cancer.

The cancer testis antigen or derivative thereof is referred to herein as the primary component of the immunotherapy.

### Tumour Antigen

In one aspect the cancer testis antigen is from the MAGE family, for example the MAGE A family, such as MAGE 3.

In one aspect the tumour antigen is an antigen selected from the following group or is an immunogenic portion or fragment thereof: WT-1, WT-1F, BAGE, LAGE 1, LAGE 2 (also known as NY-ESO-1), SAGE, HAGE, XAGE, PSA, PAP, PSCA, P501S (also known as prostein), HASH1, HASH2, Cripto, B726, NY-BR1.1, P510, MUC-1, Prostase, STEAP, tyrosinase, telomerase, survivin, CASB616, P53, and/or Her-2/neu, SSX-2; SSX-4; SSX-5; NA17; MELAN-A; P790; P835; B305D; B854; CASB618 (as described in WO00/53748); CASB7439 (as described in WO01/62778); C1491; C1584; and C1585.

The tumour or cancer testis antigen may be administered as an immunogenic protein, for example full length native protein or a chemically or genetically modified derivative thereof. Alternatively, immunogenic fragments of the protein, for example comprising 9 to 20 such as 9 to 100 amino acids may be employed.

The tumour or cancer testis antigen may be administered as a fusion protein, for example comprising protein D (or a fragment thereof) from Hepatitis B. Information on immunological fusion partner derived from protein D, can be obtained from WO 91/18926. In one embodiment, the protein D derivative comprises approximately the first ⅓ of the protein, in particular approximately the first N-terminal 100-120 amino acids such as the first 109 to 112 amino acids, more specifically the first 109 amino acids (or 108 amino acids thereof). In one embodiment, the protein D derivative may comprise amino acids 20 to 127 of protein D.

The proteins may be chemically conjugated, but are preferably expressed as recombinant fusion proteins and may allow increased levels to be produced in an expression system as compared to non-fused protein.

The fusion partner may assist in providing T helper epitopes (immunological fusion partner), preferably T helper epitopes recognised by humans, or assist in expressing the protein (expression enhancer) at higher yields than the native recombinant protein. Preferably the fusion partner will be both an immunological fusion partner and expression enhancing partner.

In one aspect the invention provides a fusion protein wherein the N-terminal portion of protein D as described herein is fused to the N-terminus of the cancer testis antigen or an immunogenic fragment thereof. More specifically the fusion with the protein D and the N-terminus of the cancer testis antigen is effected such that the cancer testis antigen replaces the C-terminal-fragment of protein D that has been excised. Thus the N-terminus of protein D becomes the N-terminus of the fusion protein.

Other fusion partners or fragments thereof may be included in fusion proteins as described herein for use in the invention in place of or in addition to protein D include, for example
the non-structural protein from influenzae virus, NS1 (hemagglutinin)—typically the N terminal 81 amino acids are utilised, although different fragments may be used provided they include T-helper epitopes,
LYTA derived from Streptococcus pneumoniae, which synthesize an N-acetyl-L-alanine amidase, amidase LYTA, (coded by the lytA gene {Gene, 43 (1986) page 265-272} such as the repeat portion of the Lyta molecule found in the C terminal end for example starting at residue 178 such as residues 188-305.

Purification of hybrid proteins containing the C-LYTA fragment at its amino terminus has been described {Biotechnology: 10, (1992) page 795-798.

Fusion proteins for use in the present invention may include an affinity tag, such as for example, a histidine tail comprising between 5 to 9 such as 6 histidine residues. These residues may, for example be on the terminal portion of protein D (such as the N-terminal of protein D) and/or the may be fused to the terminal portion of the cancer testis antigen.

Generally however the histidine tail with be located on terminal portion of the cancer testis antigen such as the C-terminal end of the cancer testis antigen. Histidine tails are advantageous in aiding purification.

In one embodiment of the present invention the tumour antigen is protein D-MAGE-3, in which the tumour antigen comprises approximately or exactly the first 127 amino acids of protein D, with or without one or two amino acid substitutions to the sequence in which the amino acids K-2 and L-3 of protein D are replaced with the unrelated amino acids D-2 and P-3. This numbering is for the amino acid sequence of protein D including the 18 aa signal sequence. In one embodiment, the protein D-MAGE-3 antigen does not include the 18 amino acid signal sequence of protein D. The antigen may include one or two linker amino acids before the protein D sequence and the MAGE-3 sequence. The antigen may further comprise an optional His tail, for example a 7-aa His tail. In this embodiment, the antigen may comprise one or two linker amino acids between the MAGE-3 sequence and the His tail. In one embodiment, the following sequence may be used (SEQ ID NO:1):

| | |
|---|---|
| MDPKTLALSLLAAGVLAGCSSHSSNMANTQMKSDKIIIAH | 40 |
| RGASGYLPEHTLESKALAFAQQADYLEQDLAMTKDGRLVV | 80 |
| IHDHFLDGLTDVAKKFPHRHRKDGRYYVIDFTLKEIQSLE | 120 |
| MTENFETMDLEQRSQHCKPEEGLEARGEALGLVGAQAPAT | 160 |
| EEQEAASSSSTLVEVTLGEVPAAESPDPPQSPQGASSLPT | 200 |
| TMNYPLWSQSYEDSSNQEEEGPSTFPDLESEFQAALSRKV | 240 |
| AELVHFLLLKYRAREPVTKAEMLGSVVGNWQYFFPVIFSK | 280 |
| ASSSLQLVFGIELMEVDPIGHLYIFATCLGLSYDGLLGDN | 320 |
| QIMPKAGLLIIVLAIIAREGDCAPEEKIWEELSVLEVFEG | 360 |
| REDSILGDPKKLLTQHFVQENYLEYRQVPGSDPACYEFLW | 400 |
| GPRALVETSYVKVLHHMVKISGGPHISYPPLHEWVLREGE | 440 |
| EGGHHHHHHH | 451 |

UNDERLINE = first 127 amino acids of Protein
D including 18 amino acid sequence
NO UNDERLINE = unrelated amino acids and His
tail
Asp-Pro substituted within Protein D sequence
Met-Asp at aa 128-129 to create a cloning site))
Gly-Gly at 442-443)
7 his tail
DOUBLE UNDERLINE = fragment of MAGE3; amino
acids 3-314 of MAGE3 (312 aas total)

The immunotherapy may comprise mixture of one or more tumour specific or cancer testis antigens, and/or one or more peptides thereof, and/or or more fusion proteins thereof.

Alternatively, vectors comprising DNA encoding for the protein or an immunogenic fragment thereof may be administered.

An immune response may generated against the vector carrying the encoding DNA and thus the general immune response may be boosted (ie the vector is itself acting as an adjuvant).

The immunotherapy may, for example be administered as a prime boost regime.

### Adjuvants

When the term “adjuvant” is used in this specification in relation to a component of the immunotherapy it will generally relate to an agent which boosts the patients immune response to the primary component of the immunotherapy.

Such adjuvants are well known in the art and can be administered in a separate formulation or may be a component of the formulation comprising the primary component of the immunotherapy.

In one embodiment of the present invention the adjuvant is or comprises an aluminium salt such as aluminium hydroxide gel (alum) or aluminium phosphate, or may also be a salt of calcium, iron or zinc, or may be an insoluble suspension of acylated tyrosine, or acylated sugars, cationically or anionically derivatised polysaccharides, or polyphosphazenes. In a further embodiment of the invention the adjuvant is or comprises CpG containing oligonucleotides, for example oligonucleotides characterised in that the CpG dinucleotide is unmethylated. Such oligonucleotides are well known and are described in, for example WO 96/02555.

In the formulation of the inventions it may be desirable that the adjuvant composition induces an immune response preferentially of the TH1 type. In one embodiment, the adjuvant for use in the present invention may include, for example a combination of monophosphoryl lipid A, preferably 3-de-O-acylated monophosphoryl lipid A (3D-MPL) together with an aluminium salt. CpG oligonucleotides may also preferentially induce a TH1 response.

In one embodiment, the present invention may comprise an adjuvant system that involves the combination of a monophosphoryl lipid A and a saponin derivative, for example a combination of QS21 and 3D-MPL as disclosed in WO 94/00153, or a less reactogenic composition where the QS21 is quenched with cholesterol as disclosed in WO 96/33739.

In one embodiment, the adjuvant formulation may comprise QS21, 3D-MPL & tocopherol, for example, in an oil in water emulsion is described in WO 95/17210.

In an alternative embodiment, the adjuvant formulation may comprise QS21, 3D-MPL & CpG or equivalent thereof or CpR in an oil in water emulsion or in a liposomal formulation.

In one embodiment, the adjuvant for the immunotherapy may be or comprise a TLR 7, 8 or 9 agonist, such as a TLR 9 agonist.

### Chemotherapy Agents

Suitable chemotherapeutic agents for use in the combination therapy of the invention include:
alkylating agents, for examples derived from platins such as cisplatin (for example 80 mg/m² intravenously, for example over 1-2 hours), carboplatin or oxaliplatin,
plant alkaloids such as vincristine, vinblastine, vinorelbine (for example 30 mg/m²), vindesine,
terpenoids such as paclitaxel or docetaxel,
fluracil,
gemcitabine (for example 1250 mg/m² intravenously for example over 30 mins),
treatments derived from isoflavones such as phenoxidiol, and
combinations of any of the above such as cisplatin and gemcitabine or vinorelbine.

Thus in one aspect the chemotherapeutic used in the chemotherapy is selected from taxol, cisplatin, gemcitabine, vinorelbine or a multiple signal transduction regulator such as phenoxodiol.

More specifically gemcitabine may; for example, be administered as 1250 mg/m² intravenously over approximately 30 minutes; vinorelbine may for example, administered as 30 mg/m² intravenously over approximately 30 minutes; or cisplatin may, for example, administered as 80 mg/m² intravenously over approximately 1-2 hours.

The cisplatin may, for example be administered approximately 4 hours following the infusion of one of the other chemotherapeutic agents such as gemcitabine or vinorelbine.

Other suitable chemotherapeutic agents for use in the present invention include dacarbazine, which is currently approved for the treatment of metastatic malignant melanoma and non-Hodgkin's lymphoma, melphalan (trade name Alkeran), temozolomide, carmustine employed in the treatment of brain tumors, non-Hodgkin's lymphoma, melanoma and multiple myeloma, and tamoxifen.

In one embodiment of the present invention the chemotherapeutic agent is cyclophosphamide.

### Corticosteroids/Anti-Emesis Agents

In one embodiment of the present invention, the methods or combinations as described herein may further comprise or include administration of corticosteroids and/or anti-emesis medicaments such as ondansetron or dexamethasone. These may be administered in support of chemotherapy, as appropriate. In one embodiment of the present invention, the methods or combinations as described herein may further comprise or include administration or inclusion of dexamethasone. The corticosteroid or anti-emesis agent, for example dexamethasone, may be administered prior to or concurrently with chemotherapeutic agents as described herein or with an immunotherapy as described herein.

In one embodiment, the corticosteroid or anti-emesis agent, for example dexamethasone, may be administered 8, 7, 6, 5, 4, 3, 2 or 1 day prior to or concurrently with the immunotherapy as described herein. In one embodiment, the corticosteroid or anti-emesis agent, for example dexamethasone, may be administered 8, 7, 6, 5, 4, 3, 2 or 1 day prior to or concurrently with the chemotherapy as described herein.

The invention also extends to use of a cancer testis antigen or immunogenic derivative thereof in the manufacture of an immunotherapeutic medicament for the treatment of cancer following surgery to remove some or all a cancer (with lymph node sampling or complete lymph node dissection) and wherein said immunotherapy comprises an adjuvant and is optionally administered concomitantly or subsequent to chemotherapy or radiotherapy.

The immunotherapy may, for example be administered, in a regime commencing with a vaccination:
approximately 4 weeks after surgery, or
less than 4 weeks after a first line treatment such a chemotherapy or radiotherapy, has commenced or has been completed,
concurrently/concomitantly with chemotherapy
   with a further vaccination every subsequent third week. This may continue, for example for 10 or more weeks such as 15, 16, 20 or more weeks.

Maintenance immunotherapy can be continued as appropriate, for example with one or more vaccinations every six to twelve months.

In a further aspect the invention provides the use of a cancer testis antigen or immunogenic derivative thereof in the manufacture of an immunotherapeutic medicament for the treatment of cancer following chemotherapy and/or radiotherapy, wherein the immunotherapeutic medicaments is optionally concomitantly administered with a chemotherapeutic agent and/or radiotherapy.

In yet a further aspect the invention provides the use of a cancer testis antigen or immunogenic derivative thereof in the manufacture of an immunotherapeutic medicament for the treatment of cancer for use prior to one or more subsequent treatments for the said cancer.

Concurrently or concomitantly in the context of this specification is intended to mean administration at of the two therapies simultaneously ie the first therapy (or the effects thereof) is still in the patients system (ie has not been metabolised, excreted or the like). Thus concurrently/concomitantly includes where the two or more therapies are administered by different routes and/or at different times. In one embodiment, the terms concurrently and concomitantly are taken to mean “on the same day” or “within 1 or 2 hours”. In one embodiment, the terms concurrently and concomitantly are taken to mean “within 30 minutes”.

The terms concurrently and concomitantly are optionally substitutable throughout the specification with each other as required.

The invention also extends to kits comprising the different components of the combination therapy according the invention.

The invention also includes use of an immunotherapy including compositions thereof based on a cancer testis antigen in a combination therapy for cancer.

### EXAMPLES

### Example 1

Evaluation of the impact of pre-treatment with cyclophosphamide (CY) on the depletion of CD4+CD25+ Treg, on the immune response induced by the MAGE-A3 AS15 ASCI and on the therapeutic effect of ASCI in the TC1Mage3 therapeutic model.

### Experimental Protocol

Groups of CB6F1 female mice (n=10/group) received at day 0 sub-cutaneous (SC) 10e5 TC1-Mage3 cells followed by injection of:
Group 1: PBS at day 3-7-11-15
Group 2: MAGE-A3 (1 μg)/As 15 ASCI at day 3-7-11-15
Group 3: Cyclophosphamide (CY) (2 mg IP) at day 0+PBS at day 3-7-11-15
Group 4: CY+MAGE-A3 (1 μg)/AS15 ASCI

### Read-Outs

verification of the depletion by FACS at day 3 and 15 on whole blood
in vivo tumor growth from day 0 to day 28
ICS at day 28 on spleen cells
serology at day 28 (not completed)

### Impact of Cyclophosphamide on Lymphocyte and Treg Counts

In order to analyze the impact of 2 mg of CY injected intraperitoneally in mice, 3 and 15 days after the injection blood was taken and lymphocytes count was performed by FACS

3 days
Total Cell count (multisizer)

| Group | ×10e6 cells/ml |
|---|---|
| PBS | 2.7 |
| MAGE-3 AS15 ASCI | 2.5 |
| Cyclophosphamide (Cy) | 1 |
| MAGE-3 AS15 ASCI + Cy | 0.9 |

The injection of CY even at a low dose such as 2 mg has an impact on the total number of T cells

Treg count (FACS staining: - CD25APC
(clone PC61) - CD4PE- FoxP3FITC)

| | CD4+ | % CD4+ | |
|---|---|---|---|
| | CD25+ | CD25+/ | % Treg / |
| Group | (events) | total cell | total cell |
| PBS | 229 | 0.11 | 0.09 |
| MAGE-3 AS15 ASCI | 326 | 0.12 | 0.09 |
| Cyclophosphamide (Cy) | 274 | 0.1 | 0.07 |
| MAGE-3 AS15 ASCI + Cy | 231 | 0.09 | 0.06 |

At the concentration of 2 mg, Cyclophosphamide only slightly decreased the number of T reg but did not deplete them.

15 days
Total Cell count (multisizer)

| Group | ×10e6 cells/ml |
|---|---|
| PBS | 7.53 |
| MAGE-3 AS15 ASCI | 4.39 |
| Cyclophosphamide (Cy) | 5.53 |
| MAGE-3 AS15 ASCI + Cy | 3.31 |

15 days after the injection of CY, the total number of T cells is almost restored

Treg count (FACS staining: - CD25APC
(clone PC61) - CD4PE- FoxP3FITC)

| | CD4+ | % CD4+ | |
|---|---|---|---|
| | CD25+ | CD25+/ | % Treg/ |
| Group | (events) | total cell | total cell |
| PBS | 613 | 0.21 | 0.16 |
| MAGE-3 AS15 ASCI | 308 | 0.1 | 0.09 |
| Cyclophosphamide (Cy) | 539 | 0.16 | 0.13 |
| MAGE-3 AS15 ASCI + Cy | 633 | 0.08 | 0.06 |

15 days after cyclophosphamide injection the % of T reg remained lower in the group of mice receiving the ASCI+cyclophosphamide.

### Impact of Cyclophosphamide on the Immune Response Induced by MAGE-A3 AS15 ASCI.

14 days after the last ASCI injection, the T cell response was analyzed by intracellular cytokine staining of CD4 and CD8 using flow cytometry (3 pools per group) after a short in vitro restimulation (2 Hrs) with the pool of MAGE-A3 overlapping peptide spanning the entire MAGE-A3 sequence.

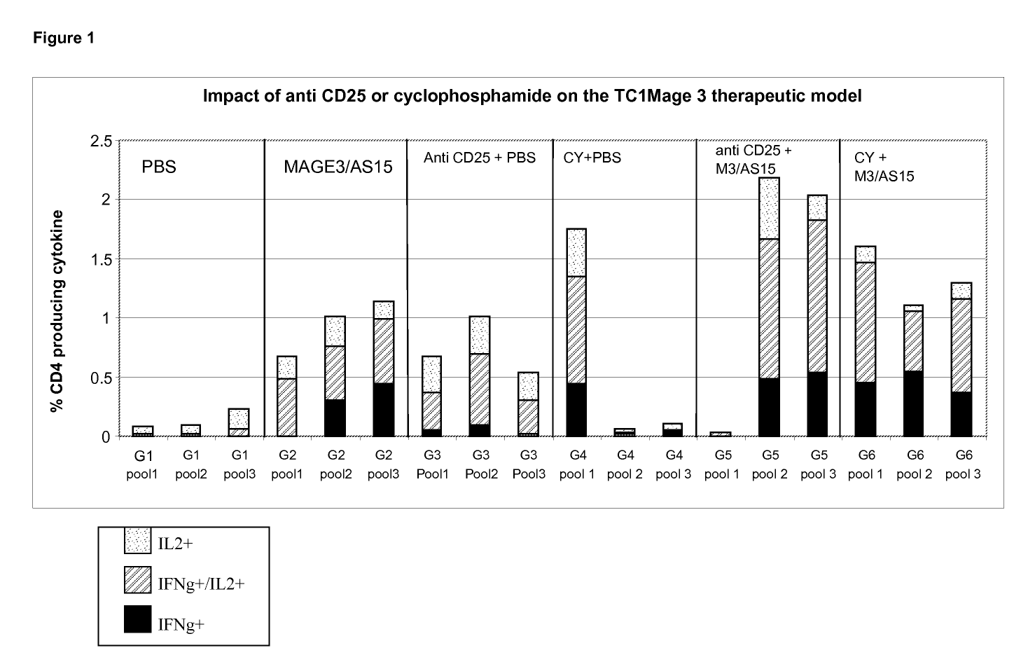

FIG. 1 shows the CD4 response.
NB There appears to be an inversion of G4 pool 1 and G5 pool 1
Cyclophosphamide by itself did not induce any MAGE-A3 specific CD4 response.
Injection of cyclophosphamide before the ASCI injection did not abolish the CD4 response induced by the ASCI there is even a trend in favor of a better response when the cyclophosphamide is given 3 days before the ASCI.

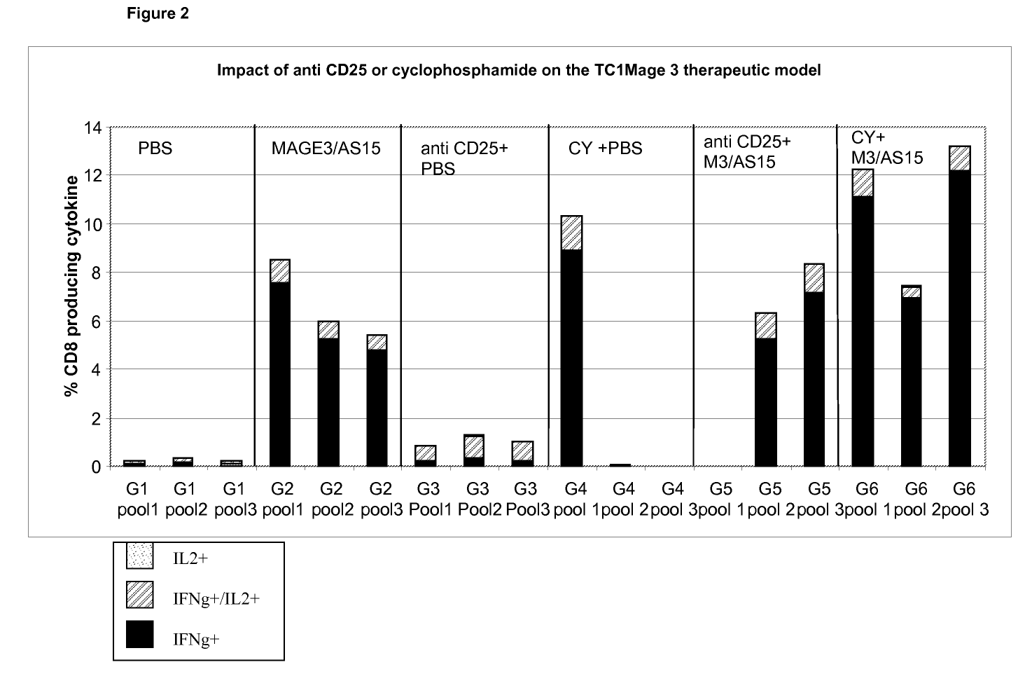

FIG. 2 shows the CD8 response
NB There appears to be an inversion of G4 pool 1 and G5 pool 1
Cyclophosphamide by itself did not induce any MAGE_A3 specific CD8 response.
Injection of cyclophosphamide before the ASCI injection did not abolish the CD8 response induced by the ASCI there is even a trend in favor of a better response when the cyclophosphamide is given 3 days before the ASCI.
   Therapeutic Effect of Cyclophosphamide: Combination with ASCI

Groups of 10 mice were challenged at day 0 with 10e5 TC1 MAGE3 cells injected subcutaneously. MAGE-A3 AS15 ASCI are injected at day 3, 7, 11, 15+/−a pre-treatment with cyclophosphamide at day 0.

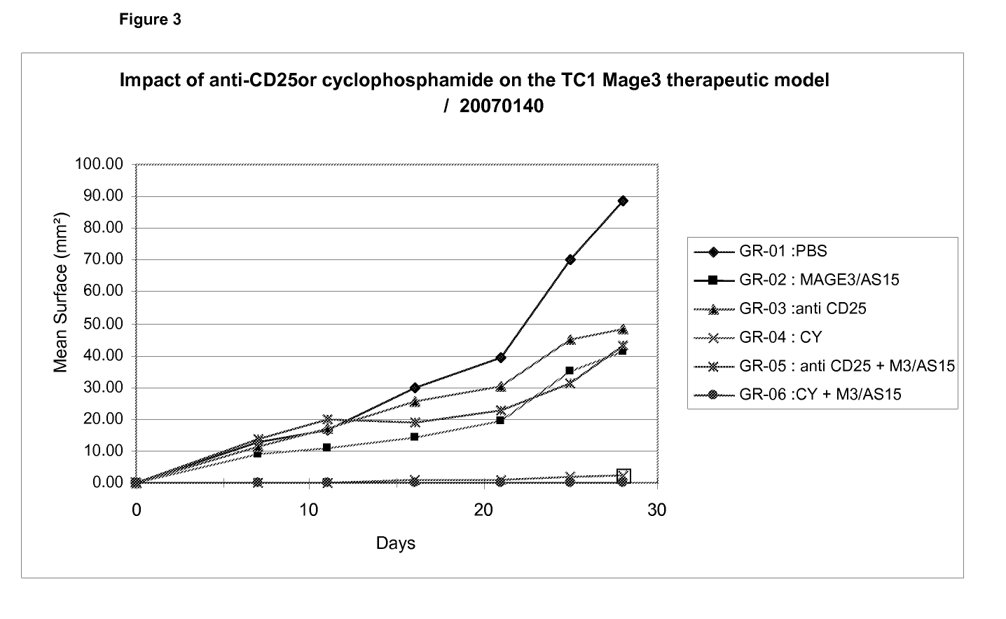

Individual tumor growth was followed over time for 4 weeks and the mean tumor growth per group of 10 animals is reported.
FIG. 3 shows In vivo tumor growth of TC1Mage3 cells from day 0 to day 28 (10e5 cells injected)

### Conclusion:

100% of the animals receiving PBS develop growing tumors.
Injection of MAGE-A3 AS15 ASCI slows down the tumor growth
One single injection of 2 mg cyclophosphamide at the day of tumor challenge is sufficient to induce tumor rejection in 100% of the animals.
The combination of the ASCI also gives rise to 100% tumor rejection.

### Example 2

Evaluation of whether pre-treatment with dexamethasone could negatively impact the immune response induced by MAGE-A3+AS15 ASCI and the capacity of ASCI to protect mice against a tumor challenge.

A series of 3 experiments (exp 20060590-20060803-20070129) were conducted in mice to evaluate the effect of doses (10, 20, 50 mg/Kg) of dexamethasone on
the total number and phenotype of spleen cells and PBL,
the impact of dexamethasone on humoral and cellular immune responses induced by MAGE-A3+AS15 ASCI+/−dexamethasone and
the capacity of ASCI+/−dexamethasone to protect mice against a tumor challenge.

The schedule of injection of dexamethasone mimics more or less the human situation with cycles of chemotherapy which would be spaced by 3 weeks and dexamethasone given before each cycle.

ASCI are given in the days following dexamethasone injection at the time patients would have received their chemotherapy.

The doses of dexamethasone used in these mice experiments (10-50 mg/kg) were previously described to have an impact on the immune system in mice.

The analysis of the immune response was performed 1, 7 or/and 14 days after the second ASCI injection both on spleen cells and peripheral blood lymphocytes (PBL).

The read out performed were:
Lymphocyte/cell count (multisizer)
Extracellular staining for surface marker (CD4, CD8, B, NK cells) and analysis by flow cytometry (FACS)
Intracellular cytokine staining (ICS) by FACS
Serology (ELISA)
Tumor protection

### Experimental Protocol

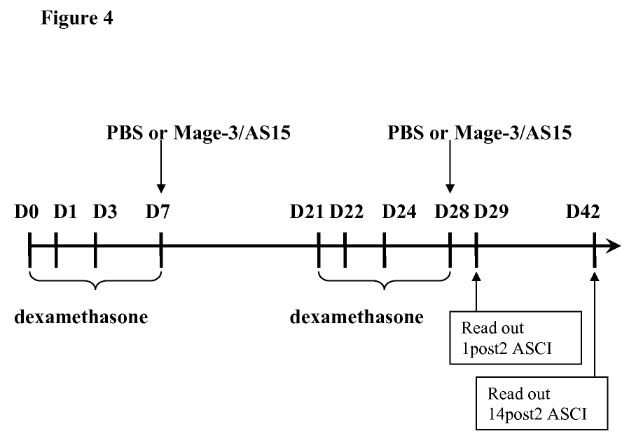

Depending on the experiment, Groups of CB6F1 mice received one or the other of the following treatments (see FIG. 4):
PBS
Dexamethasone 20 mg/kg
MAGE-A3 (10 μg)/AS15 ( 1/10) IM
MAGE-A3 (10 μg)/AS15 ( 1/10) IM+dexamethasone 10 mg/kg-50 mg/kg IP

### Impact of Dexamethasone on the Total Lymphocyte Count in Spleen and PBL

The total number of lymphocytes was analyzed in the different groups 1, 7 or/and 14 days after the second injection of ASCI+/−Dexamethasone.

The data from the 3 experiments show that in the spleen of animals receiving Dexamethasone at doses between 10 to 50 mg/kg, there is a decrease of the total lymphocyte count detected one day after the ASCI injection. This effect is transient as 7 and 14 days after the ASCI, the lymphocyte count has recovered.

In the PBL the data appear to be inconsistent and more difficult to interpret, but it seems that the recovery of the lymphocyte count is slower in PBL and is not complete 14 days after the second ASCI (18 days after the last injection of Dexamethasone)

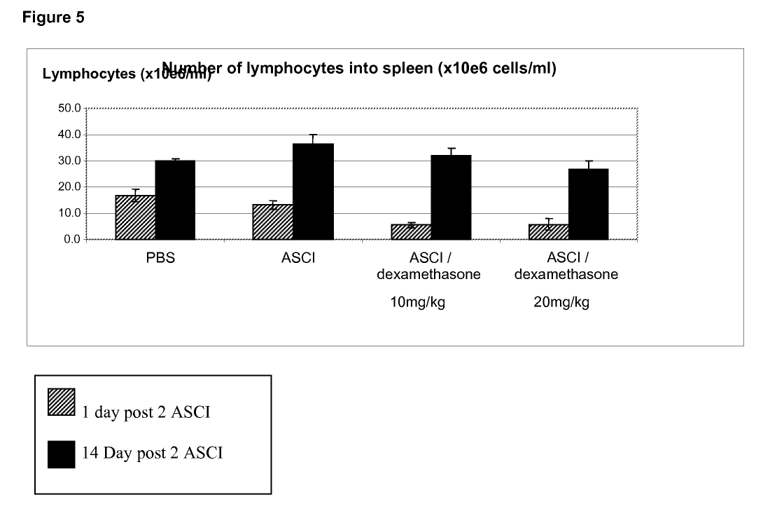

FIG. 5—Exp 20060590: n=3 mice/group individually treated

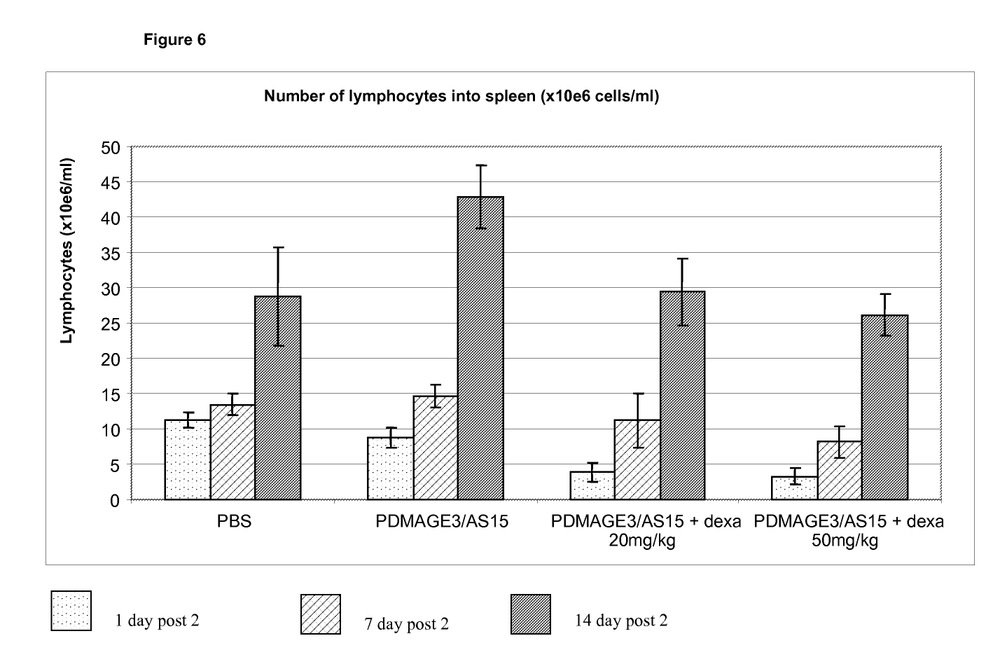

FIG. 6—Exp 20060803 n=3 mice/group individually treated)

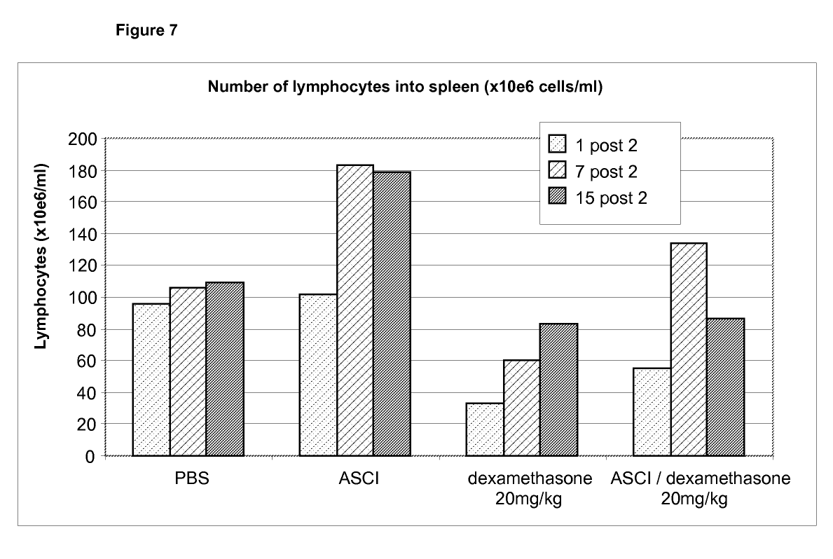

FIG. 7—Exp 20070129 n=3 mice/group individually treated

### Impact of Dexamethasone on the Different Cell Subpopulations in Spleen

Whatever the timepoint (1, 7 or 14 days) after the last dexamethasone injection, there is no change in the relative % of CD4-CD8 T cells, B cells or NK cells in the spleen or in the PBL. Even though there is a decrease in the total lymphocyte count, all cell sub-population are affected similarly. Similar data are obtained in PBL (not shown)

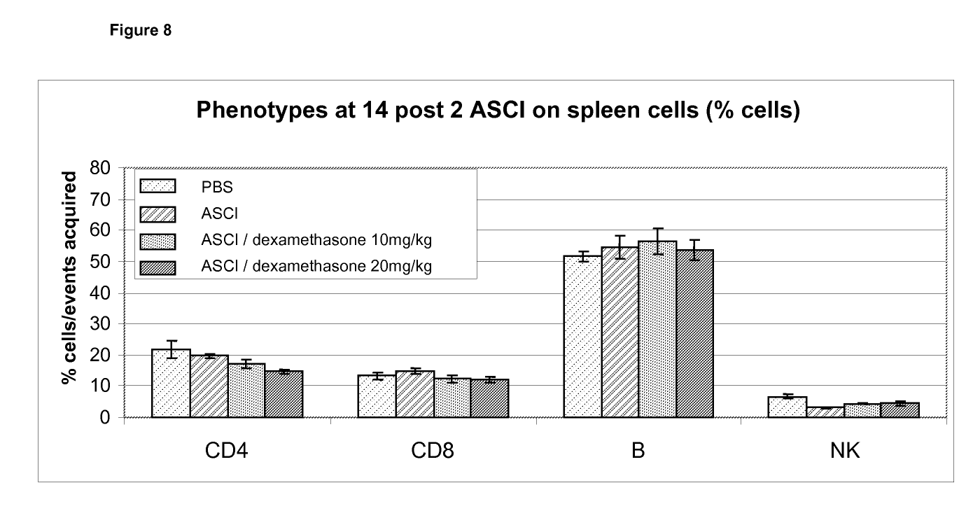

FIG. 8—Exp 20060590 n=3 mice/group individually treated

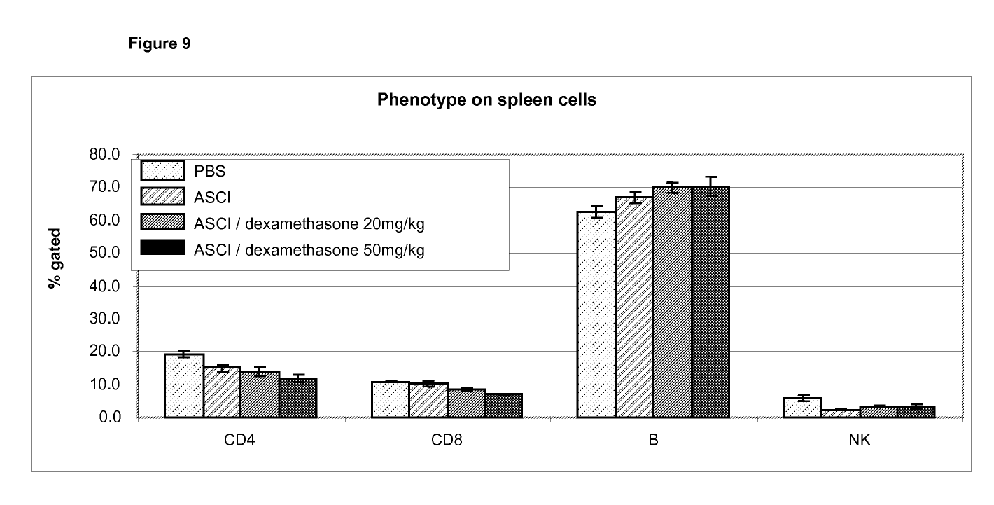

FIG. 9—Exp 20060803 n=3 mice/group individually treated

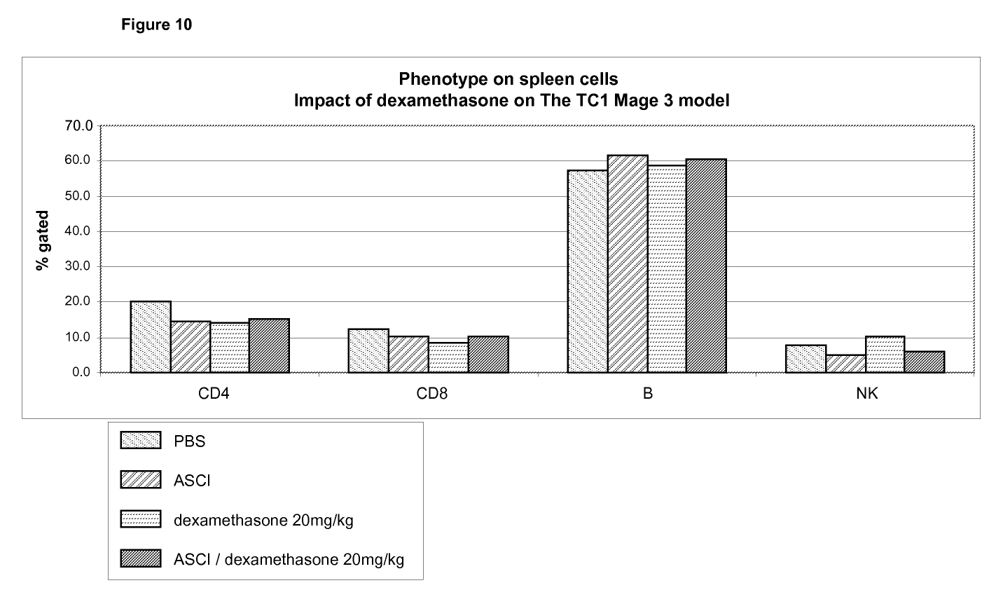

FIG. 10—Exp 20070129 n=3 mice/group individually treated

### Impact of Dexamethasone on the CD4 and CD8 T Cell Response Induced by the MAGE-A3+AS15 ASCI in Spleen and PBL

The data obtained from the 3 independent experiments, show that although there is some variability from experiment to experiment, on the contrary to what was expected there is no major negative impact of dexamethasone on the ability of the ASCI to induce T cells which produce cytokines.
The CD4 response induced by the MAGE-A3+AS15 ASCI in the presence of dexamethasone is slightly lower or equal to the response induced by the MAGE-A3 AS15 ASCI in 2 out of 3 experiments,
The CD8 response induced by the MAGE-A3+AS15 ASCI in the presence of dexamethasone is on the contrary slightly higher or equal to the response induced by the MAGE-A3 AS15 ASCI in 2 out of 3 experiments.

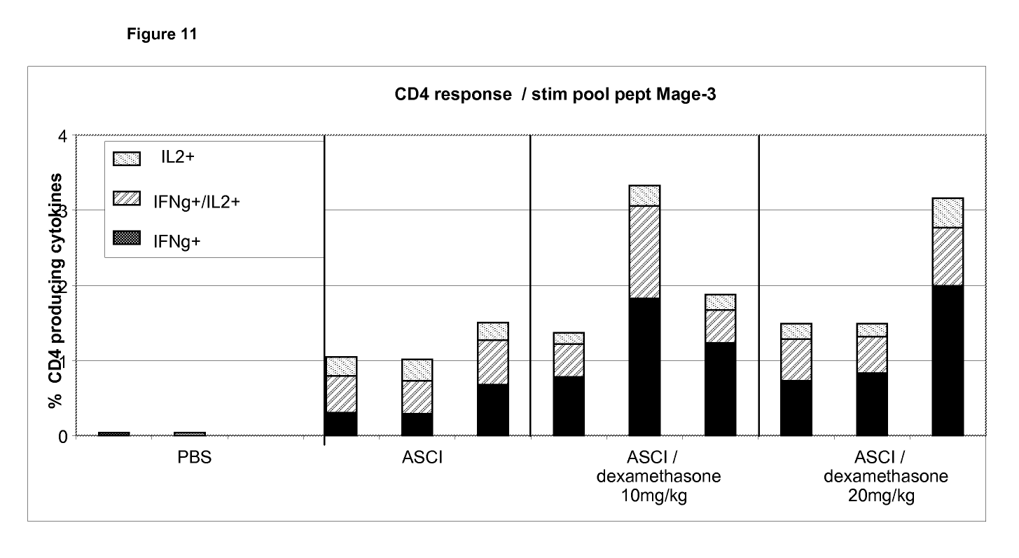

FIG. 11—Exp 20060590: CD4 response in spleen (n=3 mice/group individually treated)

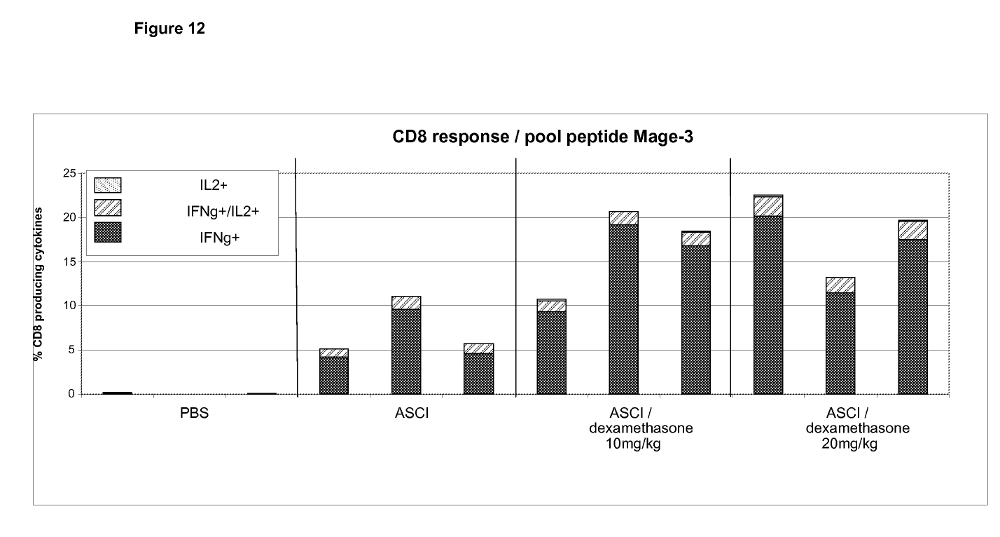

FIG. 12—Exp 20060590: CD8 response in spleen (n=3 mice/group individually treated)

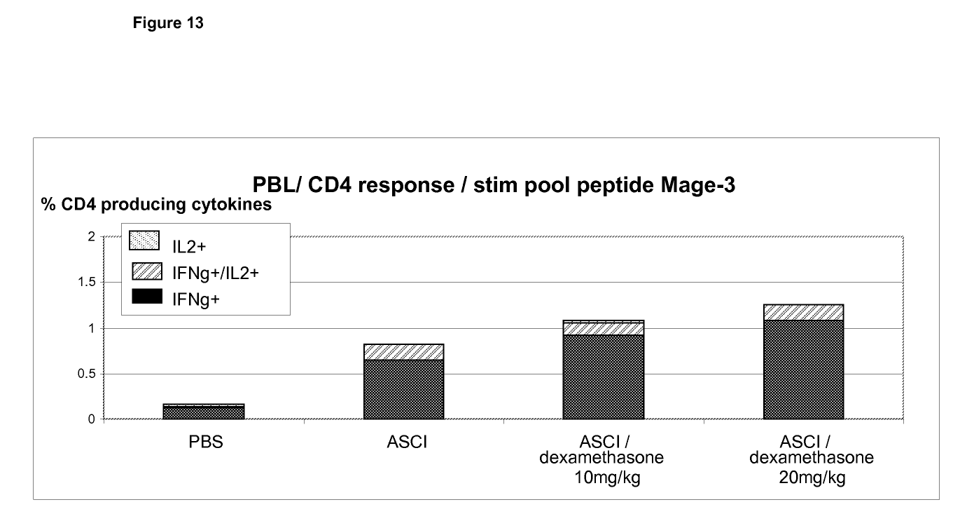

FIG. 13—Exp 20060590: CD4 response in PBL (1 pool/group of 3 mice)

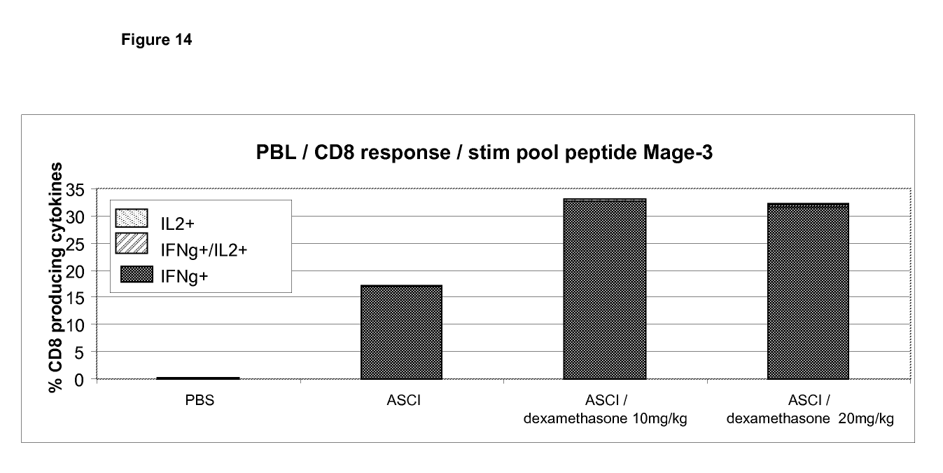

FIG. 14—Exp 20060590: CD8 response in PBL (1 pool/group of 3 mice)

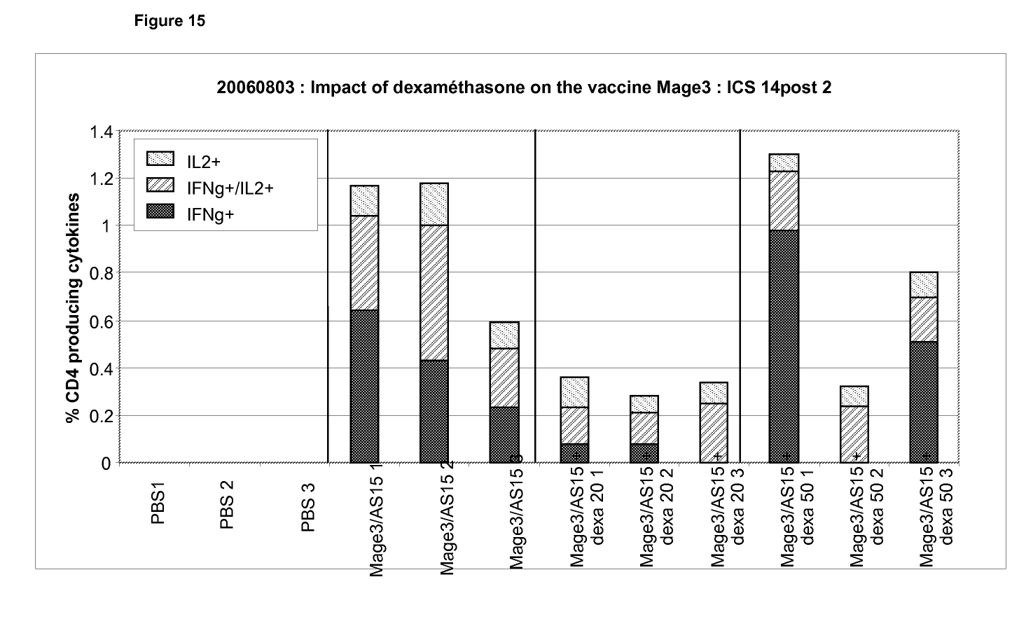

FIG. 15—Exp 20060803: CD4 response in spleen (n=3 mice/group individually treated)

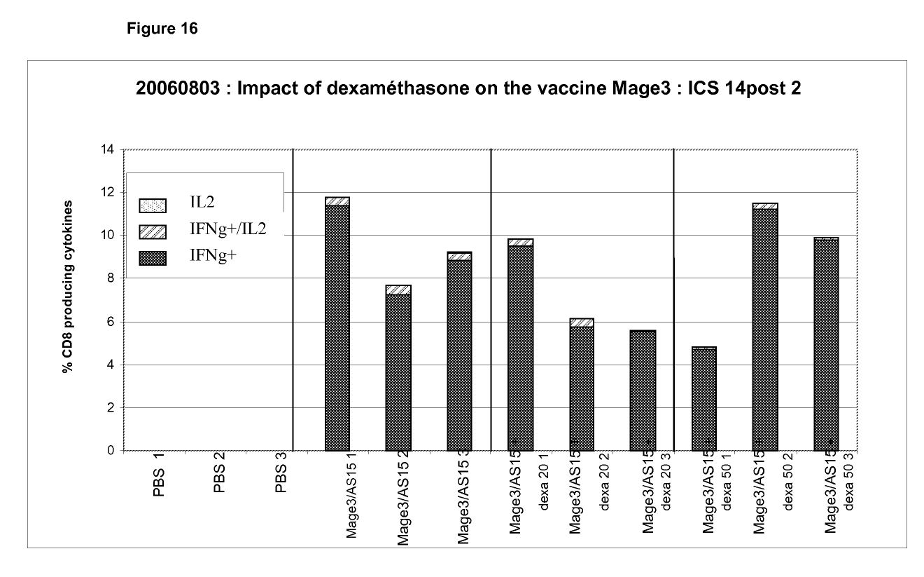

FIG. 16—Exp 20060803: CD8 response in spleen (n=3 mice/group individually treated)

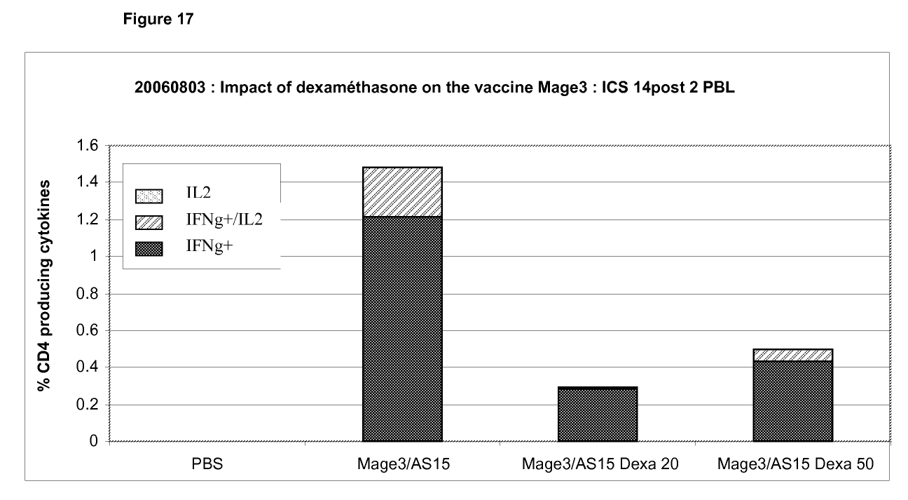

FIG. 17—Exp 20060803: CD4 response in PBL (1 pool of 3 mice/group)

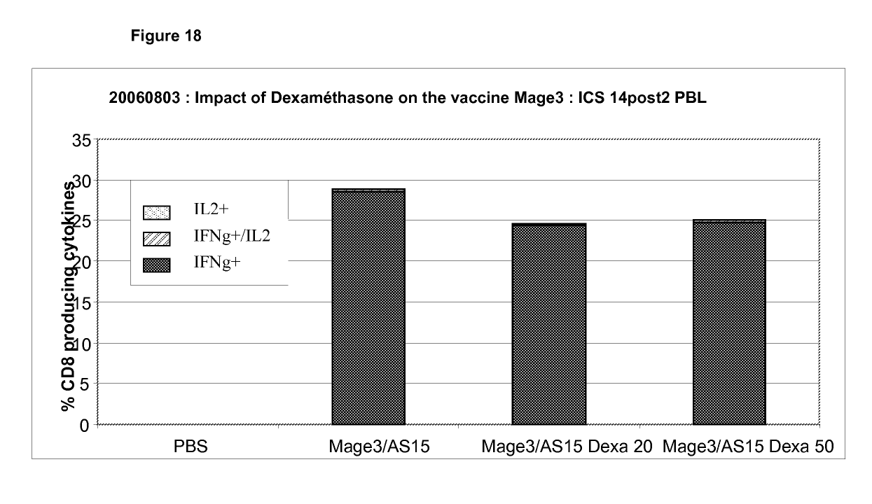

FIG. 18—Exp 20060803: CD4 response in PBL (1 pool of 3 mice/group)

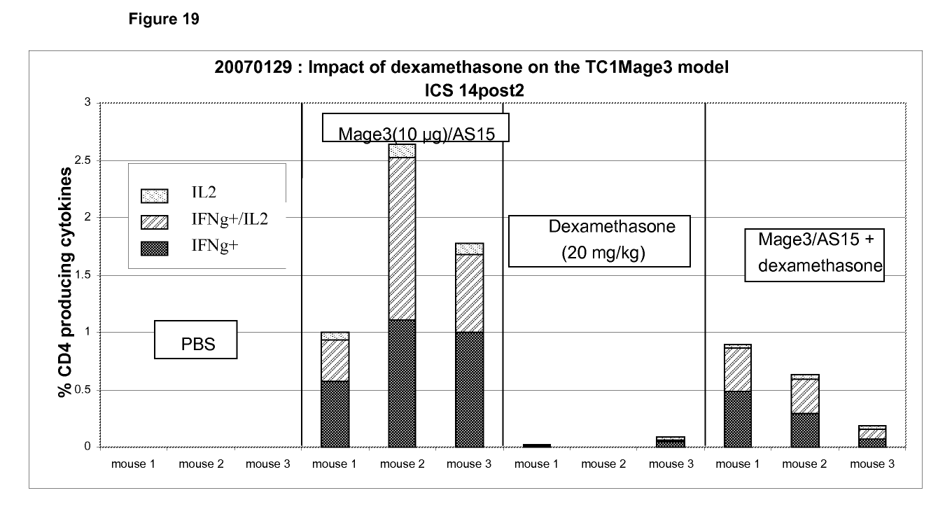

FIG. 19—Exp 20070129: CD4 response in spleen (n=3 mice/group individually treated)

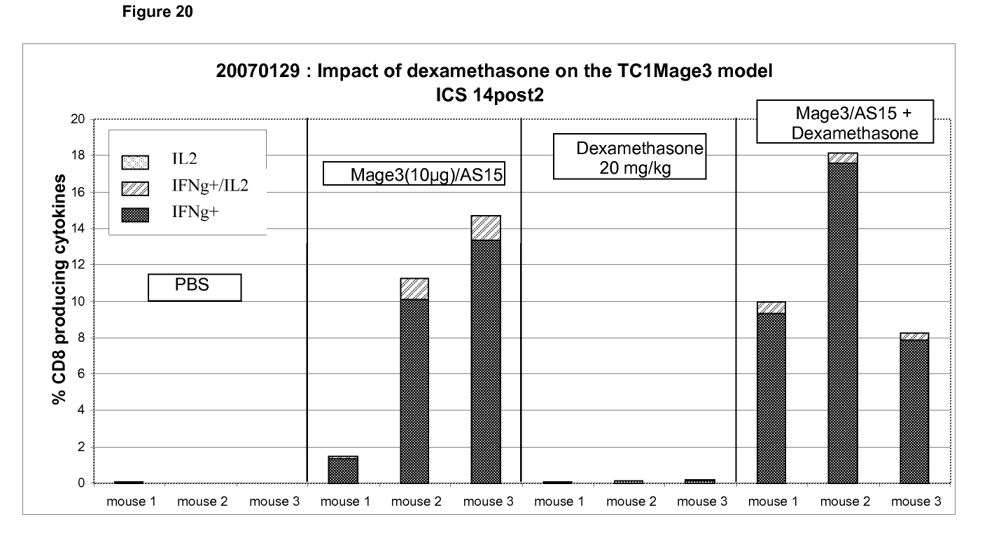

FIG. 20—Exp 20070129: CD8 response in spleen (n=3 mice/group individually treated)

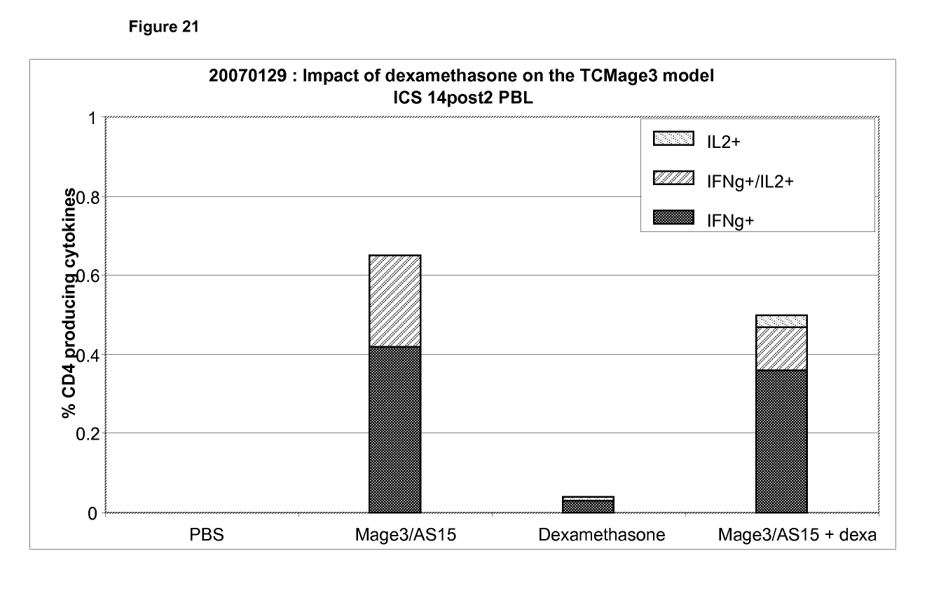

FIG. 21—Exp 20070129: CD4 response in PBL (1 pool of 3 mice/group)

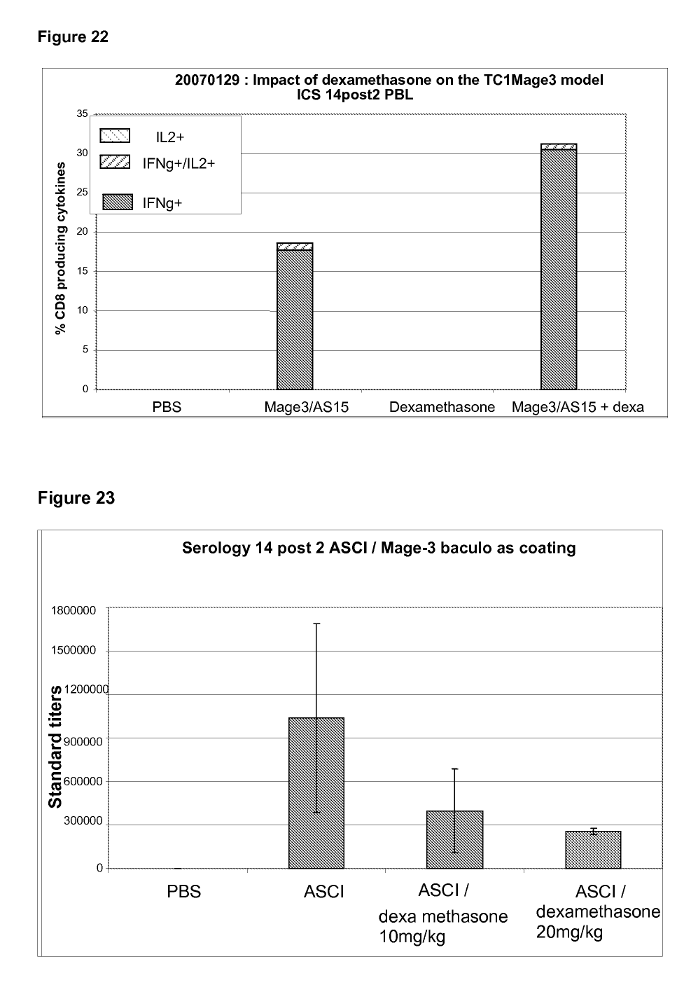

FIG. 22—Exp 20070129: CD8 response in PBL (1 pool of 3 mice/group)

### Impact of Dexamethasone on the Antibody Response Induced by the MAGE-A3+AS15 ASCI.

The antibody response was analyzed in one experiment (exp 20060590) 14 days after the last ASCI injection and 18 days after the last dexamethasone injection (see FIG. 23)

The data show that there is a clear decrease in the antibody titer induced by the MAGE-A3 AS15 ASCI in the presence of dexamethasone.

This is in agreement with the observation made in PBL: the total lymphocyte count did not seem to recover as quickly as in the spleen.

n=3 mice/group Individually treated

### Impact of Dexamethasone on the Capacity of MAGE-A3+AS15 ASCI to Protect Mice Against a Tumor Challenge.

In order to evaluate if a pre-treatment with dexamethasone could be detrimental for the anti-tumor potential of ASCI, groups of 11 mice were immunized twice at 3 weeks interval (day 7 and 28) dexamethasone is given several times, in the week before each ASCI injection.
Group 1: PBS
Group 2: MAGE-A3+AS15 ASCI
Group 3 Dexamethasone alone
Group 4: MAGE-A3+AS15 ASCI+Dexamethasone

2 weeks after the last immunization, mice were challenged with 10e6 murine tumor cells expressing MAGE-A3 (TC1-MAGE3 cells).

Individual tumor growth is recorded twice a week for 4 weeks and the data are expressed as the mean tumor growth per group of 11 animals.

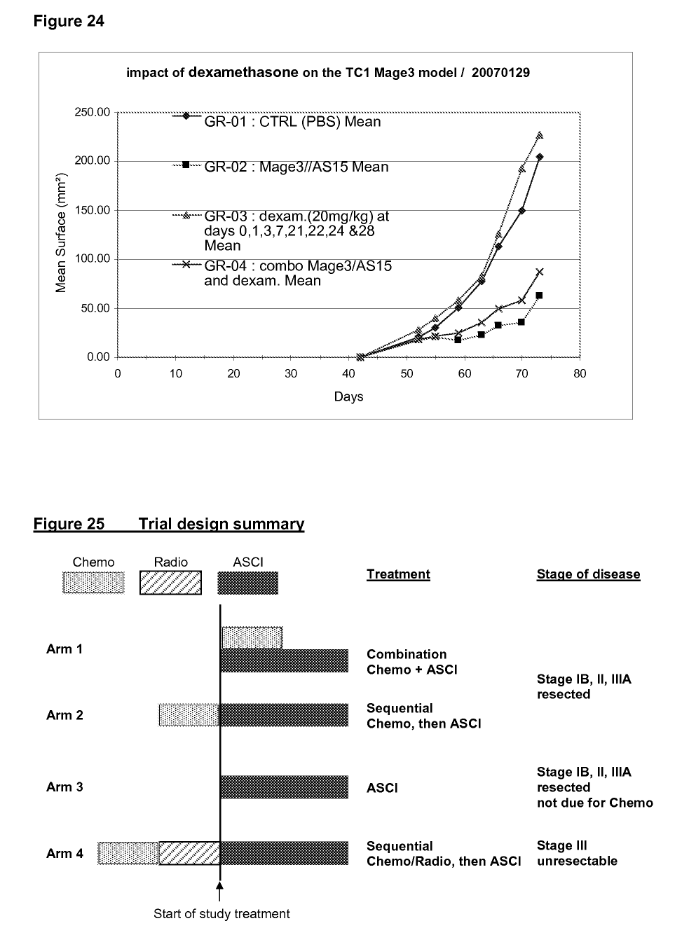

As shown on FIG. 24, all the mice which received either PBS or dexamethasone alone develop a tumor.
Tumor which received the ASCI are partially protected against the tumor challenge
The pre-treatment with dexamethasone before each ASCI injection have no major impact on the tumor protection. This is in agreement with the absence of impact seen on the immune response induced.

In conclusion:

### Impact of Dexamethasone on the Total Lymphocyte Count and Cell Sub-Population

Dexamethasone lead to a transient depletion in total lymphocyte count in spleens of immunized animals, this without affecting one cell (T, B, NK) in particular.

### Impact of Dexamethasone on the CD4 and CD8 T Cell Response Induced by the MAGE-A3+AS15 ASCI in Spleen and PBL

On the contrary to what was expected, there is no major negative impact of dexamethasone on the ability of the ASCI to induce T cells which produce cytokines.
The CD4 response induced by the MAGE-A3+AS15 ASCI in the presence of dexamethasone is slightly lower or equal to the response induced by the MAGE-A3 AS15 ASCI while
The CD8 response is on the contrary slightly higher or equal to the response induced by the MAGE-A3 AS15 ASCI

### Impact of Dexamethasone on the Antibody Response Induced by the MAGE-A3+AS15 ASCI.

The antibody titer reached after 2 injections of MAGE-A3 AS15 ASCI is decreased in the presence of dexamethasone.

### Impact of Dexamethasone on the Capacity of MAGE-A3+AS15 ASCI to Protect Mice Against a Tumor Challenge.

The protective effect induced by the ASCI is conserved after a pre-treatment with dexamethasone.

### General Conclusion:

Taken together, these data show that although a negative impact on the antibody response is seen, a pre-treatment with active doses of dexamethasone given before the injection of an ASCI should not be problematic as
no negative impact on the T cell response, the main component of the anti tumor effect is observed. Moreover,
the protective effect induced by the ASCI is conserved after a pre-treatment with dexamethasone.

### Example 3

A Phase I/II study is in progress to assess the safety and immunogenicity of recMAGE-A3+AS15 cancer immunotherapeutic given as adjuvant therapy, with or without adjuvant chemo(-radio)therapy, to patients with MAGE-A3-positive Non-Small Cell Lung cancer (stage I, II or III)

Adult patients with pathologically proven MAGE-A3-positive non-small-cell lung cancer in stage IB, II or III, will be included. Inclusion criteria will be cohort-dependent and will ensure that each patient's state and planned treatment are appropriate for the cohort in question.

Patients in three distinct populations will be included:
i) Patients with resected stage IB, II or IIIA tumors who are due for chemotherapy, for admission to Cohorts 1 and 2,
ii) Patients with resected stage IB, II or IIIA tumors who are not due for chemotherapy, for admission to Cohort 3,
iii) Patients with unresectable stage III tumors, following chemotherapy and/or radiotherapy, for admission to Cohort 4.

The diagram in FIG. 25 summarises the four treatment arms in this study.

This is an open, four-arm, parallel-group study. All patients will receive the same immunotherapeutic treatment, but they will be recruited into four cohorts from the respective populations defined above:
Cohort 1: Patients with resected stage IB, II or IIIA tumors who are due for chemotherapy. These patients will receive chemo- and immunotherapy in parallel.
Cohort 2: Patients with resected stage IB, II or IIIA tumors who are due for chemotherapy. These patients will first receive chemotherapy and then, after completion of chemotherapy, they will receive immunotherapy.
Cohort 3: Patients with resected stage IB, II or IIIA tumors who are not due for chemotherapy. These patients will receive immunotherapy.
Cohort 4: Patients with unresectable stage III tumors, following chemotherapy and/or radiotherapy. These patients will receive immunotherapy.

Immunotherapeutic treatment will comprise eight doses of the recombinant antigen recMAGE-A3 combined with the immunological adjuvant system AS15. AS15 comprises 3D-MPL, QS21 and CpG in a liposomal formulation. Doses will be administered at three-week intervals; in Cohort 1 this may be adapted to fit in with the patient's chemotherapy.

Chemotherapy and radiotherapy will be based on standard practice, as follows:
In Cohort 1, chemotherapy will comprise 2-4 cycles of cisplatin (CDDP, 80 mg/m², cycle day 1) plus vinorelbine (30 mg/m², cycle days 1 and 8).
In Cohort 2, cisplatin and vinorelbine will previously have been administered according to the usual treatment procedures at the investigation site.
In Cohort 3, no chemo- or radiotherapy will have been administered before or during the MAGE-A3 administration.
In Cohort 4, radiotherapy and chemotherapy before the study will follow the site's usual treatment procedures.

During the study (i.e. MAGE-A3 administration), adjuvant radiotherapy is allowed in Cohorts 1, 2 and 3 for patients in stage III only and is prohibited in Cohort 4. Chemotherapy during the study is allowed in Cohort 1 only as described above, and is prohibited in Cohorts 2-4.

The total maximum duration of the study for a patient will be 30-35 weeks, depending upon the cohort.

### Adjuvant Chemo- and Radiotherapy

For Cohort 1 the administration of recMAGE-A3+AS15 ASCI will be done on the eighth day of each cycle of chemotherapy. This day has been selected in order to avoid a concomitant administration with corticosteroids (as anti-emetic treatment) usually administered on the first day of each cycle (CDDP+vinorelbine) or the need for patients to make an additional visit to the hospital (i.e. fourteenth day of each cycle).

What is claimed is:

1. A method of treating cancer comprising administering to a patient with a cancer a therapeutically effective amount of an immunotherapeutic composition comprising a tumour antigen, and an adjuvant, prior to surgical resection of the cancer, where surgical resection includes removal of lymph nodes.

2. A method according to claim 1, wherein the tumor antigen is a MAGE antigen selected from the group consisting of MAGE A1, A2, A3, A4, A5, A6 and/or MAGE C1 and/or C2.

3. A method according to claim 2, wherein the tumor antigen is MAGE-A3.

4. A method according to claim 1, in which the tumor antigen is in the form of a fusion protein further comprising a fusion partner selected from the group consisting of: protein D from Haemophilus influenzae B, a fragment of H. influenzae Protein D comprising amino acids 1 to 109 of protein D, a fragment of H. influenzae Protein D comprising amino acids 20 to 127 of protein D, and a fragment of H. influenzae Protein D comprising amino acids 1 to 127 of protein D.

5. A method according to claim 4, in which the fusion protein comprises MAGE-A3 or an immunological fragment thereof comprising amino acids 3-314 of MAGE-A3.

6. A method according to claim 1, wherein the surgery is total resection of the cancer.

7. A method according to claim 1, wherein the cancer is melanoma, breast cancer, prostate cancer, bladder cancer, ovarian cancer, lung cancer, non-small cell lung carcinoma (NSCLC), head and neck cancer, seminoma or liver cancer.

8. A method according to claim 1, wherein the cancer is NSCLC.

9. A method according to claim 1, wherein the adjuvant comprises a CpG containing oligonucleotide.

10. A method according to claim 1, wherein the adjuvant is in a liposomal formulation.

11. A method according to claim 1 wherein the adjuvant comprises 3D-MPL or QS21.

12. A method according to claim 1, further comprising the administration of dexamethasone.

13.-28. (canceled)

29. A method of treating a MAGE A3 positive Non-small Cell Lung Cancer (NSCLC), comprising administering to a patient with MAGE A3 positive Non-small Cell Lung Cancer (NSCLC) a therapeutically effective amount of an immunotherapeutic composition comprising a MAGEA3 tumor antigen and an adjuvant, said administration prior to surgical resection of the cancer, and where surgical resection includes removal of lymph nodes.

30. A method according to claim 29, where said surgical resection includes complete lymph node dissection.

31. A method according to claim 29, further comprising chemotherapy.

32. A method according to claim 29, further comprising radiotherapy.

33. A method according to claim 29, further comprising administration of dexamethasone.

34. A method of treating a MAGE A3 positive Non-small Cell Lung Cancer (NSCLC), comprising administering to a patient with MAGE A3 positive Non-small Cell Lung Cancer (NSCLC) a therapeutically effective amount of an immunotherapeutic composition comprising a MAGEA3 tumor antigen and an adjuvant, said administration concurrent with or following surgical resection of the cancer, and where surgical resection includes removal of lymph nodes.


SEQUENCE LISTING (2 sequences)

SEQ ID NO: 1 - protein, 450 aa, Artificial Sequence
  fusion of protein D-MAGE-A3
MDPKTLALSLLAAGVLAGCSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMDLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGGHHHHHHH

SEQ ID NO: 2 - protein, 85 aa, MAGEArtificial Sequence
  MAGE Core signature sequence - consensus
LIXVLXXIXXXGXXAPEEXIWEXLXXMXXXXGXEXXXXGXPXXLLTXXXVQEXYLXYXQVPXSXPXXYEFLWGPRAXXETXXXKV